(19) United States
(12) Patent Application Publication
(10) Pub. No.: US 2002/0146755 A1
(43) Pub. Date: Oct. 10, 2002

(54) Quality control material

(76) Inventors: Toshiyuki Baba, Hyogo (JP); Takashi Fukui, Hyogo (JP); Yuzzo Hasegawa, Hyogo (JP); Hisahide Hiura, Hyogo (JP); Yahiro Uemura, (US)

Correspondence Address: SUGHRUE, MION, ZINN, MACPEAK & SEAS, PLLC, 2100 PENNSYLVANIA AVENUE, N.W., WASHINGTON, DC, 20037-3213, US

(21) Appl. No.: 09/774,616

(22) Filed: Feb. 1, 2001

(51) Int. Cl. C12Q 1/48

(52) U.S. Cl. 435/15; 436/15

(57) ABSTRACT

A quality control material composition or a standard material for clinical laboratory tests, containing recombinant human serum albumin prepared by recombinant technology as a base component.

### BACKGROUND OF THE INVENTION

1. Field of the Invention

The present invention relates to a quality control material or a standard material for use in clinical laboratory tests.

2. Description of Related Art

Quality control materials or standard materials for use in clinical laboratory tests are prepared by adding various enzymes, proteins or serum components of human or animal origin to human or animal sera or bovine serum albumin solutions.

Recently, in place of enzymes, proteins and serum components of human or animal origin, there are known those quality control materials or standard materials prepared by adding enzymes, proteins or the like prepared by recombinant technology to human or animal sera or bovine serum albumin solutions. The quality control material or standard materials prepared by such formulations tend to undergo fluctuation among lots or have the problem that they are not completely free of infection when they are prepared based on human or animal sera.

On the other hand, in the case of those quality control materials or standard materials prepared based on bovine serum albumin, the impurities contained in bovine serum albumin are not constant, thus causing an lot to lot difference or the impurities render target enzymes, proteins or the like unstable. Also, in the case of those quality control materials or standard materials prepared based on animal sera or bovine serum albumin, the base material are not of human origin so that a difference from actual serum sample has come into problem.

Therefore, a quality control material or standard material based on a stable human type material having a smaller lot-to-lot difference is demanded.

### SUMMARY OF THE INVENTION

Therefore, an object of the present invention is to provide a quality control material or standard material based on a stable human type material having a small lot to lot difference.

The present inventors have made intensive investigation and as a result they have found that use of recombinant human serum albumin prepared by recombinant technology enables one to produce a precision control material or standard material which is superior in stability, specificity, etc. to those produced based on natural type conventional materials. The present invention is based on this discovery.

Accordingly, the present invention provides the following.

1. A quality control material composition or a standard material for clinical laboratory tests, containing recombinant human serum albumin prepared by recombinant technology as a base component.

2. The composition or the material as described in above 1, further containing at least one of enzyme, protein and serum components, each being of human or animal origin, or prepared by recombinant technology.

3. The composition or the material as described in above 2, wherein the enzyme is at least one selected from the group consisting of aspartate aminotransferase (AST), alanine aminotransferase (ALT), lactate dehydrogenase (LDH), creatine kinase (CK), alkaline phosphatase (ALP), choline esterase (CHE), leucine aminotransferase (LAP), acid phosphatase (ACP), amylase (AMY), γ-glutamyl transpeptidase (GGT), lipase (LIP) and aldolase (AL).

4. The composition or the material as described in above 2, wherein the protein is at least one selected from the group consisting of globulin (G), C reactive protein (CRP), lipoprotein (LP), transferrin (TF), ferritin (FER), hormones, and carcinoembryonic antigen.

5. The composition or the material as described in above 1, 2, 3, or 4 wherein the composition contains about 0.1 to about 20% by weight/volume of recombinant human albumin.

6. The composition or the material as described in above 1, 2, 3, or 4 wherein the composition contains about 4 to about 8% by weight/volume of recombinant human albumin.

7. A stabilizing method of a quality control material composition or a standard material for clinical laboratory tests, characterized by adding recombinant human serum albumin prepared by recombinant technology as a base component.

8. The method as described in above 7, further containing at least one of enzyme, protein and serum components, each being of human or animal origin, or prepared by recombinant technology.

9. The method as described in above 8, wherein the enzyme is at least one selected from the group consisting of aspartate aminotransferase (AST), alanine aminotransferase (ALT), lactate dehydrogenase (LDH), creatine kinase (CK), alkaline phosphatase (ALP), choline esterase (CHE), leucine aminotransferase (LAP), acid phosphatase (ACP), amylase (AMY), γ-glutamyl transpeptidase (GGT), lipase (LIP) and aldolase (AL).

10. The method as described in above 8, wherein the protein is at least one selected from the group consisting of globulin (G), C reactive protein (CRP), lipoprotein (LP), transferrin (TF), ferritin (FER), hormones, and carcinoembryonic antigen.

11. The method as described in above 7, 8, 9, or 10, wherein the amount of adding is about 0.1 to about 20% by weight/volume of recombinant human albumin.

12. The method as described in above 7, 8, 9, or 10, wherein the amount of adding is about 4 to about 8% by weight/volume of recombinant human albumin.

13. A stabilized clinical assay method using a quality control material composition or a standard material for clinical laboratory tests, characterized by adding recombinant human serum albumin prepared by recombinant technology as a base component.

14. The method as described in above 13, further containing at least one of enzyme, protein and serum components, each being of human or animal origin, or prepared by recombinant technology.

15. The method as described in above 14, wherein the enzyme is at least one selected from the group consisting of aspartate aminotransferase (AST), alanine aminotransferase (ALT), lactate dehydrogenase (LDH), creatine kinase (CK), alkaline phosphatase (ALP), choline esterase (CHE), leucine aminotransferase (LAP), acid phosphatase (ACP), amylase (AMY), γ-glutamyl transpeptidase (GGT), lipase (LIP) and aldolase (AL).

16. The method as described in above 14, wherein the protein is at least one selected from the group consisting of globulin (G), C reactive protein (CRP), lipoprotein (LP), transferrin (TF), ferritin (FER), hormones, and carcinoembryonic antigen.

17. The method as described in above 13, 14, 15, or 16, wherein the amount of adding is about 0.1 to about 20% by weight/volume of recombinant human albumin.

18. The method as described in above 13, 14, 15, or 16, wherein the amount of adding is about 4 to about 8% by weight/volume of recombinant human albumin.

The above and other objects, effects, features and advantages of the present invention will become more apparent from the following description of preferred embodiments.

### DETAILED DESCRIPTION OF THE INVENTION

Hereinafter, the present invention will be described in detail by embodiments. However, the present invention should not be construed as being limited thereto.

The rHSA of the present invention is subject to no particular limitation as long as it is a human serum albumin produced by an HSA-producing host prepared by gene manipulation. Preferred is one substantially free of contaminant components (e.g., protein) derived from a production host, more preferably one obtained by culturing an rHSA-producing host by a known means, and harvesting and purifying from culture filtrate, microorganism or cell by known separation means and purification means.

Specific examples include the following method.

(Preparation of Recombinant Human Albumin)

The host for obtaining the rHSA to be used in the present invention is subject to no particular limitation as long as it is prepared by gene manipulation. It may be disclosed in known publications or one to be developed from now. Examples thereof include microorganisms made rHSA-productive by gene manipulation (e.g., E. coli, yeast, B. subtilis and the like), animal cell, and the like. Particularly, the host is a yeast, preferably the genus Saccharomyces (e.g., Saccharomyces cerevisiae) or the genus Pichia (e.g., Pichia pastoris). An auxotrophic strain or antibiotic sensitive strain may be used. More preferably, Saccharomyces cerevisiae AH 22 strain (a, his 4, leu 2, can 1) or Pichia pastoris GTS 115 strain (his 4) is used.

A method for preparing these rHSA-producing hosts, a method for producing rHSA by culturing the host and a method for separating and harvesting the rHSA from culture may be known or analogous to a known method. For example, an rHSA-producing host can be prepared by using a typical HSA gene (EP-A-73646), (EP-A-79739), (EP-A-91527), by using a novel HSA gene (EP-A-206733), by using a synthetic signal sequence (EP-A-329127), by using a serum albumin signal sequence (EP-A-319641), by integrating a recombinant plasmid on a chromosome (EP-A-3994550, by fusing host ) EP-A-409156), by causing mutation in a methanol-containing medium, by using a mutant AOX2 promoter (U.S. Pat. Nos. 5,610,036, 5,683,893, 5,707,827 and (EP-A-506040), by expressing HSA by B. subtilis (EP-A-229712), by expressing HSA by yeast (EP-A-123544), (EP-A-248657), (EP-A-251744) or by expressing HSA by Pichia yeast (EP-A-344459).

Of these, the method causing mutation in a methanol-containing medium includes the following steps. That is, a plasmid having a transcription unit which expresses HSA under the control of an AOX1 promoter is introduced into the AOX1 gene region of a suitable host by a conventional method, preferably Pichia yeast, specifically GTS 115 strain (NRRL deposit No. Y-15851), to give a transformant (see EP-A-344459). This transformant has weak proliferation capability in a methanol medium. Therefore, this transformant is cultured in a methanol-containing medium to cause mutation and only proliferable cells are recovered. The methanol concentration here is, for example, about 0.0001-5%. The medium may be an artificial medium or natural medium. The culture conditions are 15-40 DEG C, about 1-1000 hours.

The rHSA production host is cultured by a method disclosed in the above-mentioned publications, a method wherein high concentration cells and product are obtained by fed batch culture (semi-batch culture) by supplying high concentration glucose or methanol in suitable small amounts while avoiding high concentration substrate inhibition of production cells (Japanese Patent Unexamined Publication No. 3-83595), a method wherein a fatty acid is added to the medium to enhance rHSA production (U.S. Pat. No. 5,334,512 and EP-A-504823) and the like.

The rHSA produced by culture treatment is isolated and purified at sufficient level from the components derived from the host cell and culture components by various methods. For example, a conventional method includes subjecting a yeast culture solution containing rHSA to compression→ultrafiltration membrane treatment→heat treatment→ultrafiltration membrane treatment, and further subjecting to column chromatography treatment with cation exchanger, hydrophobic chromatography treatment, column chromatography treatment with anion exchanger and the like (U.S. Pat. No. 5,440,018 and EP-A-570916), Biotechnology of Blood Proteins. 1993, vol. 227, 293-298). A method including, subsequent to the above-mentioned conventional method, a step of chelate resin treatment or a treatment of boric acid or salt thereof has been also documented (U.S. Pat. No. 5,521,287 and EP-A-612761).

Subsequent to heat treatment of this yeast culture solution, a stream line method using an adsorption fluidized bed technique (U.S. Pat. No. 5,962,649 and EP-A-699687) and the like can be also applied. The rHSA thus prepared and purified can be formulated by a known method such as sterilization by heating, ultrafiltration membrane treatment, addition of stabilizer, sterilization by filtration, dispensing, lyophilization and the like.

(Preparation of Quality Control Material Composition or Standard Material for Clinical Laboratory Tests)

Recombinant human albumin is dissolved in physiological saline or buffer, for example, Tris buffer solution in an amount of from about 0.1 to about 20% (weight/volume), more preferably from about 4 to about 8% (weight/volume) to prepare a base solution of recombinant human albumin (hereinafter, referred to as “rHSA base”). To the rHSA base may contain so-called additives such as preservatives, stabilizers, etc., if necessary or desired. The solution to be used for preparing the rHSA base may be selected from physiological saline and buffer solutions that have buffer action within desired pH ranges. The kind of buffer is not particularly limited. One or more of enzymes, proteins and serum components are added to the prepared rHSA base to prepare a quality control material or standard material. The enzyme or protein to be added includes enzymes such as aspartate aminotransferase (AST), alanine aminotransferase (ALT), lactate dehydrogenase (LDH), creatine kinase (CK), alkaline phosphatase (ALP), choline esterase (CHE), leucine aminotransferase (LAP), acid phosphatase (ACP), amylase (AMY), γ-glutamyl transpeptidase (GGT), lipase (LIP) and aldolase (AL), and proteins such as globulin (G), C reactive protein (CRP), lipoprotein (LP), transferrin (TF), ferritin (FER), hormones, carcinoembryonic antigen, and the like. The enzymes and proteins may be recombinant enzymes and proteins prepared by recombinant technology as well as natural type ones of human or animal origin. As other serum components, there may be added bilirubin (BIL), urea (BUN), uric acid (UA), calcium ion (Ca²⁺), magnesium ion (Mg²⁺), sodium ion (Na⁺), potassium ion (K⁺), lithium ion (Li⁺), iron ion (Fe²⁺), chloride ion (Cl³¹ ), lactate (LA), phosphorus (IP), glucose (Glu), creatinine (CRE), cholesterol (CHO), neutral lipid (TG), phospholipid (PL), bile acid (BA), sialic acid (SA), thyroid hormones, steroid hormones, drugs such as digoxin, antibiotics such as gentamicin and the like. These may be added singly or two or more of them may be added in admixture. They may be added in any desired concentration as far as such is within useful concentration ranges as quality control material or standard material and is not particularly limited.

The formulation prepared as described above may be filtered, divided or dispensed, or stored as it is in a liquid form, or stored by freezing or lyophilization before they can be provided as a quality control material or standard material.

### EXAMPLES

Hereinafter, the present invention will be described in more detail by examples. However, the present invention should not be construed as being limited thereto.

### Example 1

rHSA was dissolved in 10 mM Tris buffer solution (pH 7.2 ) containing 0.15 M NaCl to a concentration of 5% weight/volume to prepare a rHSA base. To this base was added an enzyme to prepare a quality control material. Immediately after the preparation and after 1 week's storage at 2 to 8° C., analyses were made. Table 1 shows the results of analyses.

TABLE 1
Quality control material prepared based on rHSA base

| | Analytical | | | | |
|---|---|---|---|---|---|
| | value | | | | |
| | immediately | | Analytical | | Residual |
| | after | | value after | | ratio |
| Item | preparation | | one week | | (%) |
| AST | 65 | IU/l | 64 | IU/l | 98.5 |
| ALT | 25 | IU/l | 25 | IU/l | 100.0 |
| LDH | 150 | IU/l | 148 | IU/l | 98.7 |
| CK | 187 | IU/l | 190 | IU/l | 101.6 |
| ALP | 156 | IU/l | 153 | IU/l | 98.7 |
| CHE | 129 | IU/l | 130 | IU/l | 101.5 |
| LAP | 40 | IU/l | 38 | IU/l | 97.5 |
| GGT | 24 | IU/l | 22 | IU/l | 91.6 |
| AMY | 75 | IU/l | 75 | IU/l | 100.0 |
| LIP | 297 | IU/l | 300 | IU/l | 101.0 |
| Na | 137 | meq/l | 135 | meq/l | 98.5 |
| K | 42 | meq/l | 40 | meq/l | 95.2 |
| Cl | 104 | meq/l | 106 | meq/l | 101.9 |
| Ca | 8.6 | mg/dl | 8.8 | mg/dl | 102.3 |
| IP | 3.1 | mg/dl | 3.0 | mg/dl | 96.8 |
| Fe | 11 | μg/dl | 108 | μ/dl | 97.3 |
| Glu | 73 | mg/dl | 74 | mg/dl | 101.4 |
| BUN | 13.8 | mg/dl | 13.0 | mg/dl | 94.2 |
| CRE | 1.25 | g/dl | 1.25 | g/dl | 100.0 |
| UA | 5.3 | mg/dl | 5.0 | mg/dl | 94.3 |
| CHO | 166 | mg/dl | 160 | mg/dl | 96.4 |
| TG | 90 | mg/dl | 92 | mg/dl | 102.2 |
| CRP | 0.6 | mg/dl | 0.5 | mg/dl | 83.3 |

### Example 2

### Comparative Example

The procedures of Example 1 were repeated except that instead of rHSA, bovine serum albumin (BSA) was used to prepare a quality control material. In the same manner as in Example 1, analyses were made immediately after the preparation and after 1 week's storage at 2 to 8° C. Table 2 shows the results of analyses.

TABLE 2
Quality control material prepared based on BSA base

| | Analytical | | | | |
|---|---|---|---|---|---|
| | value | | | | |
| | immediately | | Analytical | | Residual |
| | after | | value after | | ratio |
| Item | preparation | | one week | | (%) |
| AST | 67 | IU/l | 60 | IU/l | 89.5 |
| ALT | 25 | IU/l | 20 | IU/l | 80.0 |
| LDH | 143 | IU/l | 130 | IU/l | 90.9 |
| CK | 192 | IU/l | 180 | IU/l | 93.8 |
| ALP | 155 | IU/l | 143 | IU/l | 92.3 |
| CHE | 135 | IU/l | 130 | IU/l | 96.3 |
| LAP | 42 | IU/l | 36 | IU/l | 85.7 |
| GGT | 28 | IU/l | 21 | IU/l | 75.0 |
| AMY | 70 | IU/l | 66 | IU/l | 94.3 |
| LIP | 305 | IU/l | 290 | IU/l | 95.1 |
| Na | 150 | meq/l | 155 | meq/l | 103.3 |
| K | 36 | meq/l | 41 | meq/l | 113.9 |
| Cl | 101 | meq/l | 105 | meq/l | 104.0 |
| Ca | 8.2 | mg/dl | 8.3 | mg/dl | 101.2 |
| IP | 3.0 | mg/dl | 3.3 | mg/dl | 110.0 |
| Fe | 116 | μg/dl | 118 | μg/dl | 101.7 |
| Glu | 77 | mg/dl | 75 | mg/dl | 97.4 |
| BUN | 14.0 | mg/dl | 13.5 | mg/dl | 96.4 |
| CRE | 1.30 | g/dl | 1.25 | mg/dl | 96.2 |
| UA | 5.5 | mg/dl | 5.3 | mg/dl | 96.4 |
| CHO | 160 | mg/dl | 156 | mg/dl | 97.5 |
| TG | 85 | mg/dl | 83 | mg/dl | 97.6 |
| CRP | 0.6 | mg/dl | 0.5 | mg/dl | 83.3 |

### Example 3

### Comparative Example

The procedures of Example 1 were repeated except that instead of rHSA, human pooled serum was used to prepare a quality control material. In the same manner as in Example 1, analyses were made immediately after the preparation and after 1 week's storage at 2 to 8° C. Table 2 shows the results of analyses.

TABLE 3
Quality control material prepared based on human pooled serum

| | Analytical | | | | |
|---|---|---|---|---|---|
| | value | | | | |
| | immediately | | Analytical | | Residual |
| | after | | value after | | ratio |
| Item | preparation | | one week | | (%) |
| AST | 63 | IU/l | 47 | IU/l | 77.0 |
| ALT | 22 | IU/l | 16 | IU/l | 72.7 |
| LDH | 138 | IU/l | 125 | IU/l | 90.6 |
| CK | 186 | IU/l | 168 | IU/l | 90.3 |
| ALP | 145 | IU/l | 132 | IU/l | 91.0 |
| CHE | 138 | IU/l | 115 | IU/l | 83.3 |
| LAP | 40 | IU/l | 28 | IU/l | 70.0 |
| GGT | 25 | IU/l | 18 | IU/l | 72.0 |
| AMY | 76 | IU/l | 62 | IU/l | 81.6 |
| LIP | 315 | IU/l | 285 | IU/l | 90.5 |
| Na | 155 | meq/l | 150 | meq/l | 96.8 |
| K | 38 | meq/l | 40 | meq/l | 105.3 |
| Cl | 108 | meq/l | 111 | meq/l | 102.8 |
| Ca | 8.6 | mg/dl | 8.5 | mg/dl | 98.8 |
| IP | 3.6 | mg/dl | 4.3 | mg/dl | 119.4 |
| Fe | 126 | μg/dl | 120 | μg/dl | 95.2 |
| Glu | 81 | mg/dl | 72 | mg/dl | 88.9 |
| BUN | 13.3 | mg/dl | 13.7 | mg/dl | 105.4 |
| CRE | 1.36 | mg/dl | 1.25 | mg/dl | 91.9 |
| UA | 5.2 | mg/dl | 5.0 | mg/dl | 96.2 |
| CHO | 155 | mg/dl | 150 | mg/dl | 96.8 |
| TG | 82 | mg/dl | 83 | mg/dl | 101.2 |
| CRP | 0.5 | mg/dl | 0.4 | mg/dl | 80.0 |

### Example 4

Using the quality control materials prepared in Examples 1, 2 and 3, respectively, residual activities of AST, ALT, LAP and GGT, respectively, were measured immediately after preparation and after 1-week storage at 2 to 8° C. Table 4 shows the results.

TABLE 4
Comparison of stability of enzymes for
different base materials (2 to 8° C., 1 week storage)

| | rHSA base | BSA base | Human serum |
|---|---|---|---|
| | Residual | Residual | base |
| | ratio | ratio | Residual |
| | after 1 | after 1 | ratio |
| | week | week | after 1 week |
| | storage | storage | storage |
| Item | (%) | (%) | (%) |
| AST | 98.5 | 89.5 | 77.0 |
| ALT | 100.0 | 80.0 | 72.7 |
| LAP | 97.5 | 85.7 | 70.0 |
| GGT | 91.6 | 75.0 | 72.0 |

From the above results, it revealed that the quality control material prepared based on rHSA base was superior to those prepared based on human serum base and those prepared based on BSA base, respectively, in the stability of enzymes.

### Example 5

The quality control materials prepared in Examples 1, 2 and 3, respectively, were measured of albumin concentration by two methods, i.e., a bromocresol green method (BCG method) and a bromocresol phenol method (BCP method), respectively. Table 5 shows the results.

TABLE 5
Comparison of measured albumin concentrations
for different base materials (2 to 8° C., 1 week storage)

| | BCG Method | BCP Method |
|---|---|---|
| Kind of base | (g/dl) | (g/dl) |
| RHSA | 5.0 | 5.1 |
| BSA | 5.2 | 4.4 |
| Human serum | 5.5 | 5.4 |

From the above results, it revealed that the quality control materials prepared using BSA base showed various measured values depending on the type of assay method for albumin, which indicated that they were different in specificity. On the other hand, the quality control material prepared using rHSA base, like those using human serum base showed substantially no difference in measured values depending on the type of the assay method and showed the same level of specificity as that of human serum.

The invention may be embodied in other specific forms without departing from the spirit or essential characteristics thereof. Therefore, the present embodiment is to be considered in all respects as illustrative and not restrictive, the scope of the invention being indicated by the appended claims rather than by the foregoing description and all changes which come within the meaning and range of equivalency of the claims are therefore intended to be embraced therein.

### Example 6

### Reference Example

A Preparation Method of rHSA

(1) Heating Treatment of Culture Medium

An HSA-producing yeast Pichia pastoris was acquired and incubated in accordance with the method described in EP-A-655503.

About 2.8 liter of the culture medium including cells thus obtained was heated to 68° C. for 30 minutes as such. The heating treatment was performed in the presence of 10 mM of sodium caprylate. T his culture medium had a pH value of 6. Next, the heated solution was quickly cooled to about 15° C. and diluted about 2-fold with distilled water (total volume: 5.5 liter). Then the pH value thereof was regulated to 4.5 with an acetic acid solution.

(2) Adsorbent Particle Treatment (Streamline SP Treatment)

To a Streamline SP column (C50, 5×100 cm, gel volume; 300 ml, manufactured by Pharmacia), which had been equilibrated with a 50 mM acetate buffer (pH 4.5) containing 50 mM of sodium chloride, was fed upwardly 5.5 liter of the culture medium (electric conductivity: <10 mS) containing the yeast cells which had been obtained by the above-mentioned heating treatment (1). The feeding was made at a flow rate of 100 cm/h under stirring. Next, the same buffer (2.5 times by volume as much as the column capacity) as the one employed for the equilibration of the column was fed upwardly into the column to thereby wash the column at a flow rate of 100 cm/h for 1 hour and then at 300 cm/h for 30 minutes. Subsequently, the flow direction was reversed and an eluent [a 100 mM phosphate buffer (pH 9) containing 300 mm of sodium chloride, flow rate: 50 cm/h] was fed into the column. Thus a fraction containing rHSA was obtained.

The rHSA-containing fraction thus eluted was detected by measuring the absorbance at 280 nm.

(3) Heating Treatment

The rHSA-containing fraction thus obtained was heated at 60° C. for 1 hour in the presence of 10 mM of cysteine, 5 mM of sodium caprylate and 100 mM of aminoguanidine hydrochloride at pH 7.5.

(4) Hydrophobic Chromatography

The rHSA solution heated in the above (3) was poured into a column packed with Phenyl-Cellulofine (5×25 cm, gel volume: 500 ml, manufactured by Chisso Corporation) which had been equilibrated with a 50 mM phosphate buffer (pH 6.8) containing 0.15M of sodium chloride. Under these conditions, the rHSA was not adsorbed by the Phenyl-Cellulofine column but passed therethrough. The rHSA-containing solution passing through the column was concentrated to a volume of about 0.2 liter using an ultrafiltration membrane having a molecular weight cutoff of 30,000 (manufactured by Millipore) and the rHSA-containing solution was replaced by a 50 mM phosphate buffer (pH 6.8).

(5) Anion Exchanger Treatment

After the completion of the hydrophobic chromatography, the rHSA-containing solution, which had been concentrated and buffer-replaced, was poured into a column packed with DEAE-Sepharose FF (5×25 cm, gel volume: 500 ml, manufactured by Pharmacia) which had been equilibrated with a 50 mM phosphate buffer (pH 6.8).

Under these conditions, the rHSA was not adsorbed by the DEAE-Sepharose column but passed therethrough. The rHSA passing through the column was concentrated to a volume of about 0.2 liter using an ultrafiltration membrane having a molecular weight cutoff of 30,000 (manufactured by Millipore) and the rHSA-containing solution was replaced by distilled water.

(6) Chelate Resin Treatment

To 0.2 liter of the purified rHSA having a concentration of about 7% was added acetic acid to thereby regulate the pH value to 4.5. Then it was poured into a column packed with DIAION CRB02 (5×2.5 cm, gel volumes 500 ml, manufactured by Mitsubishi Kasei Corporation), which had been equilibrated with a 50 mM sodium acetate buffer (pH 4.5), and circulated overnight. Under these conditions, the rHSA was not adsorbed by the gel but passed through the column.

(7) Boric Acid/borate Treatment

The rHSA concentration was adjusted to 2.5%, while the electric conductivity of the solution was regulated to 1 mS or below. Sodium tetraborate was added thereto to give a final concentration of 100 mM. Next, calcium chloride was added thereto to give a final concentration of 100 mM, while maintaining the pH value at 9.5. After allowing to stand for about 10 hours, the precipitate thus formed was removed and the supernatant was recovered, concentrated and desalted. Then it was concentrated by using an ultrafiltration membrane having a molecular weight cutoff of 30,000 (manufactured by Millipore) and subjected to buffer replacement. If necessary, stabilizers (sodium caprylate and acetyltryptophan) were added followed by filter sterilization using a 0.22 m filter (manufactured by Millipore). The resulting rHSA solution can be used for injection.

(Properties of Purified rHSA)

(1) HPLC analysis

The rHSA was analyzed by means of HPLC gel filtration under the following conditions:

(a) Column: TSK gel G3000SW (Tosoh Corp.)

(b) Eluent: 0.1M KH₂PO₄/0.3M NaCl buffer

(c) Detection: absorbance at 280 nm

The purified rHSA was as a single peak of HSA monomer.

(2) Analysis of Yeast-derived Components

A culture supernatant of a yeast strain which does not produce HSA, was partially purified in accordance with the above-mentioned purification process. Rabbits were immunized with the partially purified fraction, an antiserum was obtained from the rabbits and using the antiserum, detection of yeast-derived components in the purified rHSA solution (rHSA concentration: 250 mg/ml) was carried out by means of enzyme immunoassay (EIA). The content of the yeast-derived components in the purified rHSA was 1 ng or less per rHSA 250 mg.

(3) Molecular Weight

The molecular weight was determined by the above-mentioned HPLC gel filtration method. The purified rHSA had a molecular weight of about 67,000.

(4) Isoelectric Point

The isoelectric point was determined in accordance with the method of Allen et al. [J. Chromatog., 146, 1 (1978)] with the use of a polyacrylamide gel. The purified rHSA had an isoelectric point of about 4.9.

(5) Coloring Degree of Coloring

The coloring degree was determined by using a solution of the purified rHSA (rHSA concentration: 250 mg/ml), measuring the absorbance of this solution at 280, -10 350, 450 and 500 nm and calculating the A350/A280 ratio, A450/A280 ratio and A500/A280 ratio. The purified rHSA had the coloring degree, A350/A280 of about 0.015, A450/A280 of about 0.01 and A500/A280 of about 0.002, respectively.

(6) Determination of Pyrogen

The content of pyrogen was determined by using Endospecy (Seikagaku Corporation) in accordance with the manufacture's instruction attached to the product. The content in the purified rHSA was 0.5 EU or less per rHSA 250 mg.

What is claimed is:

1. A quality control material composition or a standard material for clinical laboratory tests, containing recombinant human serum albumin prepared by recombinant technology as a base component.

2. The composition or the material as claimed in claim 1, further containing at least one of enzyme, protein and serum components, each being of human or animal origin, or prepared by recombinant technology.

3. The composition or the material as claimed in claim 2, wherein the enzyme is at least one selected from the group consisting of aspartate aminotransferase (AST), alanine aminotransferase (ALT), lactate dehydrogenase (LDH), creatine kinase (CK), alkaline phosphatase (ALP), choline esterase (CHE), leucine aminotransferase (LAP), acid phosphatase (ACP), amylase (AMY), γ-glutamyl transpeptidase (GGT), lipase (LIP) and aldolase (AL).

4. The composition or the material as claimed in claim 2, wherein the protein is at least one selected from the group consisting of globulin (G), C reactive protein (CRP), lipoprotein (LP), transferrin (TF), ferritin (FER), hormones, and carcinoembryonic antigen.

5. The composition or the material as claimed in claim 1, 2, 3, or 4 wherein the composition contains about 0.1 to about 20% by weight/volume of recombinant human albumin.

6. The composition or the material as claimed in claim 1, 2, 3, or 4 wherein the composition contains about 4 to about 8% by weight/volume of recombinant human albumin.

7. A stabilizing method of a quality control material composition or a standard material for clinical laboratory tests, characterized by adding recombinant human serum albumin prepared by recombinant technology as a base component.

8. The method as claimed in claim 7, further containing at least one of enzyme, protein and serum components, each being of human or animal origin, or prepared by recombinant technology.

9. The method as claimed in claim 8, wherein the enzyme is at least one selected from the group consisting of aspartate aminotransferase (AST), alanine aminotransferase (ALT), lactate dehydrogenase (LDH), creatine kinase (CK), alkaline phosphatase (ALP), choline esterase (CHE), leucine aminotransferase (LAP), acid phosphatase (ACP), amylase (AMY), γ-glutamyl transpeptidase (GGT), lipase (LIP) and aldolase (AL).

10. The method as claimed in claim 8, wherein the protein is at least one selected from the group consisting of globulin (G), C reactive protein (CRP), lipoprotein (LP), transferrin (TF), ferritin (FER), hormones, and carcinoembryonic antigen.

11. The method as claimed in claim 7, 8, 9, or 10, wherein the amount of adding is about 0.1 to about 20% by weight/volume of recombinant human albumin.

12. The method as claimed in claim 7, 8, 9, or 10, wherein the amount of adding is about 4 to about 8% by weight/volume of recombinant human albumin.

13. A stabilized clinical assay method using a quality control material composition or a standard material for clinical laboratory tests, characterized by adding recombinant human serum albumin prepared by recombinant technology as a base component.

14. The method as claimed in claim 13, further containing at least one of enzyme, protein and serum components, each being of human or animal origin, or prepared by recombinant technology.

15. The method as claimed in claim 14, wherein the enzyme is at least one selected from the group consisting of aspartate aminotransferase (AST), alanine aminotransferase (ALT), lactate dehydrogenase (LDH), creatine kinase (CK), alkaline phosphatase (ALP), choline esterase (CHE), leucine aminotransferase (LAP), acid phosphatase (ACP), amylase (AMY), γ-glutamyl transpeptidase (GGT), lipase (LIP) and aldolase (AL).

16. The method as claimed in claim 14, wherein the protein is at least one selected from the group consisting of globulin (G), C reactive protein (CRP), lipoprotein (LP), transferrin (TF), ferritin (FER), hormones, and carcinoembryonic antigen.

17. The method as claimed in claim 13, 14, 15, or 16, wherein the amount of adding is about 0.1 to about 20% by weight/volume of recombinant human albumin.

18. The method as claimed in claim 13, 14, 15, or 16, wherein the amount of adding is about 4 to about 8% by weight/volume of recombinant human albumin.
